(19) United States
(12) Patent Application Publication
(10) Pub. No.: US 2022/0338754 A1
(43) Pub. Date: Oct. 27, 2022

(54) DEVICES AND METHODS FOR STANDARDIZING BREATHING EFFORT

(71) Applicant: United States Government as Represented by the Department of Veterans Affairs, Washington, DC (US)

(72) Inventors: BRIAN P. LUCAS, Norwich, VT (US); GARRETT G. GRINDLE, Pittsburgh, PA (US)

(21) Appl. No.: 17/761,321

(22) PCT Filed: Sep. 18, 2020
(86) PCT No.: PCT/US2020/051426

§ 371 Date: Mar. 17, 2022

Related U.S. Application Data
(60) Provisional application No. 62/902,194, filed on Sep. 18, 2019.

(51) Int. Cl. A61B 5/08; A61M 16/20; A61M 16/08

(52) U.S. Cl. CPC A61B 5/08; A61M 16/208; A61M 16/0866

(57) ABSTRACT

Devices and methods for standardizing breathing effort in subjects are disclosed. An exemplary device has an exhaust housing having a proximal portion that defines a port and an opposed distal portion that defines inspiration and expiration pathways. The distal portion has a distal end surface, and the inspiration and expiration pathways are in fluid communication with the port and extend, respectively, to inlet and outlet openings defined in the distal end surface. An airflow adjustment plate, which defines openings having varying sizes, is rotatably coupled to the distal portion of the exhaust housing. Rotation of the airflow adjustment plate relative to the exhaust housing among a plurality of rotational positions, with a respective opening of the airflow adjustment plate being positioned in alignment with the inlet opening at each position, thereby increasing or decreasing resistance to air flow through the inlet opening.

### CROSS-REFERENCE TO RELATED APPLICATION

This application claims priority to and the benefit of the filing date of U.S. Provisional Patent Application No. 62/902,194, filed Sep. 18, 2019, which is hereby incorporated herein by reference in its entirety.

### GOVERNMENT SUPPORT CLAUSE

The subject matter in this application was developed with the funding and support of the Department of Veterans Affairs Office of Research and Development Technology Transfer Assistance Program.

### FIELD

This application relates generally to devices and methods for standardizing breathing effort in subjects, such as, for example and without limitation, subjects for whom a measurement of right atrium pressure is needed.

### BACKGROUND

An echocardiogram is an ultrasound of the internal parts of the heart. Recent reports suggest that over 7 million echocardiograms are performed on Medicare beneficiaries each year. Among these, standard transthoracic echocardiograms are the most common. Part of the reason for their popularity is that they provide a noninvasive estimate of right atrium pressure (RAP), which has important diagnostic and therapeutic implications. Most importantly, providers use RAP to manage heart failure, a common chronic condition among Medicare beneficiaries.

RAP is estimated from an echocardiogram or ultrasound by observing how the diameter of the inferior vena cava (the large vein the returns blood to the heart from the abdomen, pelvis, and legs) changes with breathing. Unfortunately this estimate often does not reflect a patient's true RAP due to measurement error. A major source of this error is how patients breathe when ultrasound images of their inferior vena cava diameters are being recorded.

Effortful breaths (high volumes of air over short periods of time) are achieved by forceful expansions of the lungs. This lowers the pressure in the chest, and as a physiologic consequence, air flow increases to the lungs. The lower pressure in the chest also lowers RAP directly, which increases the flow of blood back to the heart. A drop in RAP and an increase in blood flow back to the heart will tend to collapse the inferior vena cava. The degree of this collapse depends heavily on just how effortful the breaths are. When the effort of breathing is known, the amount of collapse of the inferior vena cava can be used to estimate the RAP.

Patients often unwittingly change their breathing efforts moment to moment. In addition, sonographers vary widely in how they instruct patients to breathe while they obtain inferior vena cava images. Some sonographers instruct patients to breathe normally (or ‘quietly’) while others ask patients to perform quick bursts of ‘nose-breathing’ called ‘sniffs’. This lack of standardization in the effort of breathing limits the value of RAP estimates obtained from echocardiograms, particularly for the end-users of the estimates: the clinicians who refer patients for echocardiograms in the first place and later make important clinical decisions based on the results.

### SUMMARY

Described herein is a device comprising an exhaust assembly and a mouthpiece assembly. The exhaust assembly has an exhaust housing and at least one selectively expandable and contractible blocking component. The exhaust housing has a distal end that defines a port and an opposed proximal end that defines a central opening and an annular space positioned radially between the central opening and an outer surface of the exhaust housing. The at least one selectively expandable and contractible blocking component is received within the annular space of the exhaust housing and coupled to the exhaust housing. The mouthpiece assembly has a mouthpiece housing and a mouthpiece. The mouthpiece housing has a proximal end that defines a port and an opposed distal end that defines a central opening and an annular space positioned radially between the central opening and an outer surface of the mouthpiece housing. The mouthpiece is positioned in fluid communication with the port of the mouthpiece housing. The proximal end of the exhaust housing is rotatably coupled to the distal end of the mouthpiece housing. Rotation of the exhaust housing relative to the mouthpiece housing selectively expands or contracts the at least one selectively expandable and contractible blocking component of the exhaust assembly to increase or decrease resistance to air flow between the annular spaces of the mouthpiece housing and the exhaust housing.

Also described herein are methods of using the device. In one exemplary method, the mouthpiece of the device is operatively positioned relative to a subject. The device can be used to deliver inspiratory air to the subject in response to inspiration of the subject that exceeds a cracking pressure threshold of an exhaust check valve of the exhaust assembly. In response to exceeding the cracking pressure threshold, the exhaust check valve flexes from a resting position to an inspiration position that permits airflow from the port of the exhaust housing to the annular space of the exhaust housing.

Also described herein is a device having a longitudinal axis and comprising an exhaust assembly. The exhaust assembly can have an exhaust housing and an airflow adjustment plate. The exhaust housing can have a proximal portion that defines a port and an opposed distal portion that defines respective inspiration and expiration pathways. The distal portion can have a distal end surface. The inspiration and expiration pathways can be in fluid communication with the port and extend, respectively, to inlet and outlet openings defined in the distal end surface. The airflow adjustment plate can be rotatably coupled to the distal portion of the exhaust housing. The airflow adjustment plate can define a first set of openings that includes a plurality of openings having varying sizes. Rotation of the airflow adjustment plate relative to the exhaust housing among a plurality of rotational positions can increase or decrease resistance to air flow through the inlet opening of the exhaust housing. At each rotational position, a respective opening of the first set of openings of the airflow adjustment plate can be positioned in alignment with the inlet opening of the exhaust housing.

### BRIEF DESCRIPTION OF THE DRAWINGS

These and other aspects of the invention will become more apparent in the detailed description in which reference is made to the appended drawings wherein:

FIG. 1 is a schematic view of an exemplary device for standardizing breathing effort as disclosed herein, with the device shown in a resting position.

FIG. 2A is a schematic view of the device of FIG. 1, shown during inspiration. FIG. 2B is a schematic view of the device of FIG. 1, shown during expiration.

FIGS. 3A-3C are schematic views of an exemplary fenestration for adjusting resistance to airflow (by increasing or decreasing the amount of blockage) within the device of FIG. 1 (e.g., between the exhaust and mouthpiece housings as further disclosed herein).

FIG. 3A depicts the fenestration in a first, seated position with minimal blockage of airflow. FIG. 3B depicts the fenestration in a second position with an intermediate amount of blockage. FIG. 3C depicts the fenestration in a third position with increased airflow blockage. Optionally, as further disclosed herein, it is contemplated that the first, second, and third positions can respectively correspond to threshold inspiratory pressures of 5 mm Hg, 10 mm Hg, and 15 mm Hg.

FIG. 4 is a perspective view of a second embodiment of a device for standardizing breathing effort as disclosed herein.

FIGS. 5A-5D depict the device of FIG. 4 in a rotational position in which minimal blockage of airflow is provided. FIG. 5A is a top view of the device. FIG. 5B is front cross-sectional view showing the device in a neutral or resting position. FIG. 5C is a front cross-sectional view showing the device during inspiration. FIG. 5D is a front cross-sectional view showing the device during expiration.

FIGS. 6A-6D depict the device of FIG. 4 in a rotational position in which an intermediate amount of blockage of airflow is provided. FIG. 6A is a top view of the device. FIG. 6B is front cross-sectional view showing the device in a neutral or resting position. FIG. 6C is a front cross-sectional view showing the device during inspiration. FIG. 6D is a front cross-sectional view showing the device during expiration.

FIGS. 7A-7D depict the device of FIG. 4 in a rotational position in which an increased blockage of airflow is provided. FIG. 7A is a top view of the device. FIG. 7B is front cross-sectional view showing the device in a neutral or resting position. FIG. 7C is a front cross-sectional view showing the device during inspiration. FIG. 7D is a front cross-sectional view showing the device during expiration.

FIG. 8 is a front perspective view of a third embodiment of device for standardizing breathing effort as disclosed herein.

FIG. 9 is a rear perspective view of the device of FIG. 8.

FIG. 10 is a front perspective view of an exhaust housing of the device of FIG. 8.

FIG. 11 is a rear view of the exhaust housing of FIG. 10.

FIG. 12 is a transparent top perspective view of the exhaust housing if FIG. 10.

FIG. 13 is a front view of an airflow adjustment plate of the device of FIG. 8.

FIG. 14 is a rear view of the airflow adjustment plate of FIG. 13.

### DETAILED DESCRIPTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, and, as such, can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an,” and “the” include plural referents unless the context clearly dictates otherwise. Thus, for example, reference to “a blocking component” or “a check valve” can include two or more such blocking components or check valves unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedent “about” or “substantially,” it is contemplated that values within up to 15%, up to 10%, up to 5%, or up to 1% (above or below) of the particularly stated value or characteristic can be included within the scope of those aspects.

As used herein, the terms “optional” or “optionally” mean that the subsequently described event or circumstance may or may not occur, and that the description includes instances where said event or circumstance occurs and instances where it does not.

The word “or” as used herein means any one member of a particular list and also includes any combination of members of that list.

As used herein, the term “subject” refers to a human or animal whose breathing effort is in need of standardization. As used herein the term “patient” refers to a human subject.

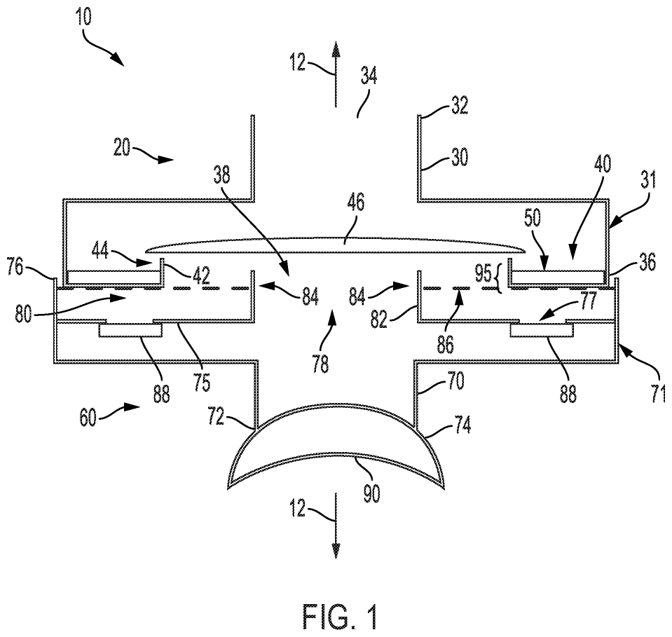

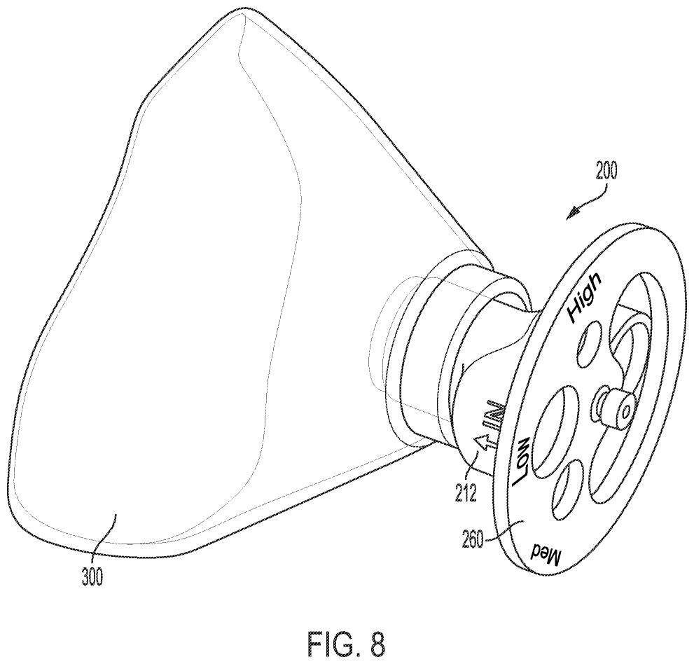

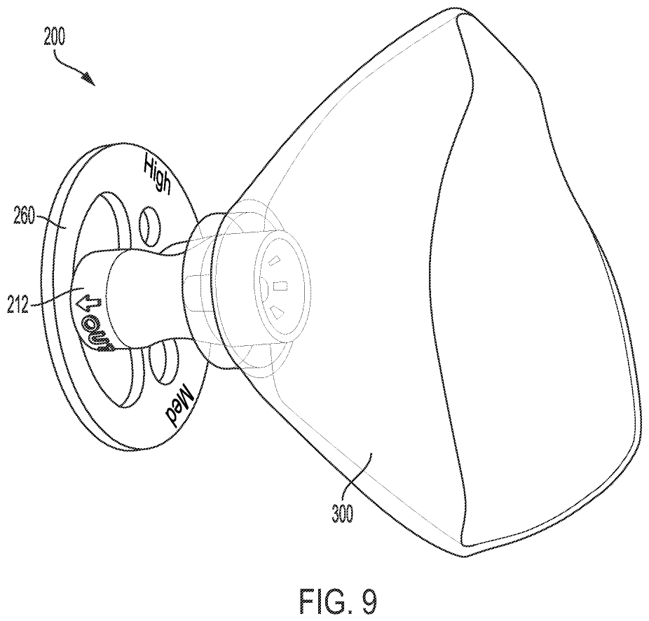

Described herein with reference to FIGS. 1-14 is a device for standardizing breathing efforts of a subject or patient. Optionally, the device can be used to standardize breathing efforts during echocardiography estimates of RAP, thereby improving the accuracy of the RAP estimates. Estimates of RAP play a key role in the diagnosis and management of chronic heart failure. Improvements in RAP reliability will also obviate re-testing due to concerns about differences in patients' breathing efforts and sonographers' instructions about how to breathe.

The disclosed devices can be hand-held and disposable, with most or all of the components made from plastic. As further disclosed herein, the device can include valves and/or deformable blocking components that provide a slight amount of resistance and are designed to open when a threshold breathing effort (inspiratory pressure) is achieved. This resistance helps to ensure that patient breathing effort does not change moment to moment. It also enhances the negative pressure in the chest. This draws blood back to the heart and causes the major vein returning blood from the lower half of the body (the inferior vena cava) to collapse. At a given respiratory effort, the amount of inferior vena cava collapse is determined by the right atrium pressure. So by knowing the amount of breathing effort used by a subject (with inspiratory resistance), and by seeing the amount of vein collapse, the disclosed device can permit accurate estimation of RAP.

The device can be adjustable so that the required threshold breathing effort can be selectively increased or decreased, depending on both the baseline breathing effort of the subject or patient and the inspiratory effort needed to cause inferior vena cava collapse. This feature is a fundamental difference between the disclosed device, which can be used for diagnostic purposes, and existing devices that are designed for therapeutic purposes.

It is contemplated that the disclosed devices can increase the validity and reliability of echocardiogram estimates of RAP. In particular, it is contemplated that echocardiogram estimates of RAP made during use of the disclosed devices can be improved in comparison to estimates of RAP made using the conventional “pulmonary artery line” measurement standard as is known in the art.

### Devices Having an Exhaust Assembly That Rotates Relative to a Mouthpiece Assembly

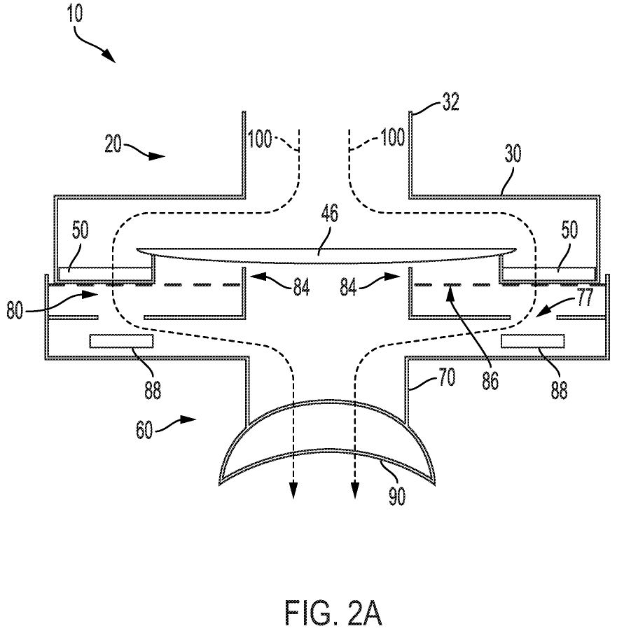

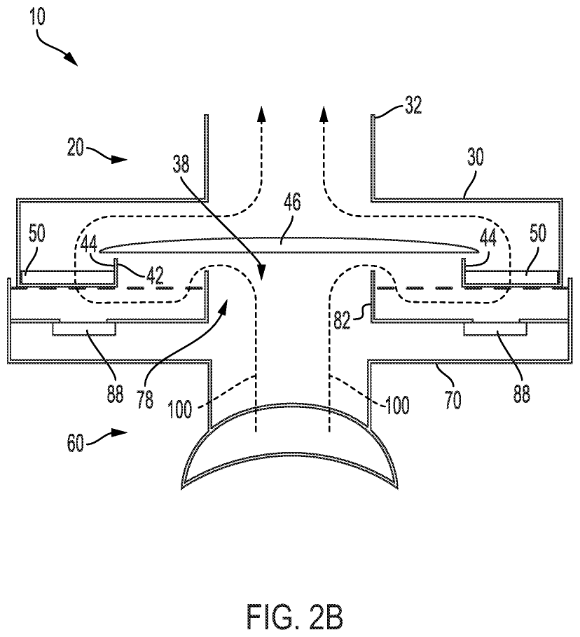

In exemplary aspects, and as shown in FIGS. 1-2B, the device 10 can comprise an exhaust assembly 20. The exhaust assembly 20 can have an exhaust housing 30. In one aspect, the exhaust housing 30 can have a distal end 32 that defines a port 34 and an opposed proximal end 36 that defines a central opening 38 and an annular space 40 positioned radially between the central opening and an outer surface 31 of the exhaust housing. The exhaust assembly 20 can further comprise at least one selectively expandable and contractible blocking component 50 received within the annular space 40 of the exhaust housing 30 and coupled to the exhaust housing.

In additional aspects, and as shown in FIGS. 1-2B, the device 10 can comprise a mouthpiece assembly 60. The mouthpiece assembly 60 can have a mouthpiece housing 70. In another aspect, the mouthpiece housing 70 can have a proximal end 72 that defines a port 74 and an opposed distal end 76 that defines a central opening 78 and an annular space 80 positioned radially between the central opening and an outer surface 71 of the mouthpiece housing. In further aspects, the mouthpiece assembly 60 can comprise a mouthpiece 90 positioned in fluid communication with the port 74 of the mouthpiece housing 70. In use, it is contemplated that the lips of a subject or patient can form a seal (e.g., a gas-tight seal) around the mouthpiece 90. Optionally, it is contemplated that a nose clip can be used to seal the nose of the subject or patient and prevent nasal breathing.

In exemplary aspects, the proximal end 36 of the exhaust housing 30 can be rotatably coupled to the distal end 76 of the mouthpiece housing 70. In these aspects, rotation of the exhaust housing 30 relative to the mouthpiece housing 70 can selectively expand or contract the at least one selectively expandable and contractible blocking component 50 of the exhaust assembly 20 to increase or decrease resistance to air flow between the annular spaces 80, 40 of the mouthpiece housing 70 and the exhaust housing 30. In further exemplary aspects, and as shown in FIG. 1, it is contemplated that the exhaust assembly 20 and the mouthpiece assembly 60 can have a common longitudinal axis 12 about which the exhaust housing 30 rotates relative to the mouthpiece housing 70.

In exemplary aspects, it is contemplated that the exhaust housing 30 and the mouthpiece housing 70 can be configured to complementary engagement, with the proximal end 36 of the exhaust housing 30 received within the distal end 76 of the mouthpiece housing 70. Optionally, in these aspects, it is contemplated that the mouthpiece housing 70 can define a circumferential ridge that retains and/or holds the exhaust housing 30 in place during rotation as disclosed herein.

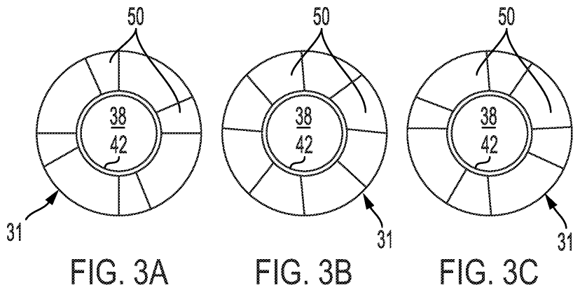

In additional aspects, the mouthpiece assembly 60 can further comprise a fenestrated layer 86 extending circumferentially within the annular space 80 of the mouthpiece housing 70. In these aspects, and as shown in FIGS. 3A-3C, expansion or contraction of the at least one selectively expandable and contractible blocking component 50 of the exhaust assembly 20 can selectively increase or decrease resistance to air flow through the fenestrated layer 86 of the mouthpiece housing 70. Optionally, in further aspects, and as shown in FIGS. 3A-3C, the blocking component 50 can comprise a plurality of fan elements. In exemplary aspects, the fenestrated layer 86 can define a plurality of openings. In further exemplary aspects, and as shown in FIG. 1, the fenestrated layer 86 can cooperate with the blocking component 50 to define an adjustable fenestration 95.

In further aspects, as shown in FIGS. 1-2B, the exhaust assembly 20 can further comprise an exhaust check valve 46 configured for movement about and between an inspiration position (FIG. 2A) and an expiration position (FIG. 2B). As shown in FIG. 2A, in the inspiration position, the exhaust check valve 46 can block airflow 100 through the central opening 78 of the mouthpiece housing 70 and direct airflow from the port 34 of the exhaust housing 30 to the annular space 40 of the exhaust housing. As shown in FIG. 2B, in the expiration position, the exhaust check valve 46 can permit airflow 100 through the central opening 38 of the exhaust housing 30. In further exemplary aspects, in the expiration position, the exhaust check valve 46 can direct airflow 100 from the central opening 38 of the exhaust housing 30 to the annular spaces 80, 40 of the mouthpiece and exhaust housings 70, 30.

Optionally, in exemplary aspects, the exhaust check valve 46 can comprise a diaphragm. During inspiration, the diaphragm can be configured to flex from its resting position in response to inspiratory pressure exceeding a cracking pressure threshold of the diaphragm. As used herein, the term “cracking pressure threshold” refers to the minimum pressure needed to flex a valve from its resting position. During expiration, the diaphragm can be configured to flex back to its rest position.

In further aspects, as shown in FIGS. 1-2B, the proximal end 36 of the exhaust housing 30 can have an annular wall 42 that defines the central opening 38 of the exhaust housing. In these aspects, the annular wall 42 can define a seat 44 configured to support the exhaust check valve 46 when the exhaust check valve is in the inspiration and expiration positions as shown in FIGS. 2A-2B.

In additional aspects, as shown in FIGS. 1-2B, the distal end 76 of the mouthpiece housing 70 can have an annular wall 82 at least partially received within the central opening 38 of the exhaust housing 30. In these aspects, when the exhaust check valve 46 is in the inspiration position as shown in FIG. 2A, the annular wall 82 of the mouthpiece housing 70 can define a stop surface 84 that is configured to contact the exhaust check valve 46 to block airflow 100 through the central opening 78 of the mouthpiece assembly 70. When the exhaust check valve 46 is in the expiration position as shown in FIG. 2B, the stop surface 84 of the annular wall 82 of the mouthpiece housing 70 can be spaced from the exhaust check valve 46 to permit airflow 100 from the central opening 78 of the mouthpiece housing 70 into the central opening 38 of the exhaust housing 30.

In exemplary aspects, and as shown in FIGS. 1-2B, the mouthpiece assembly 60 can further comprise a mouthpiece check valve 88 configured for movement about and between an inspiration position (FIG. 2A) and an expiration position (FIG. 2B). As shown in FIG. 2B, in the expiration position, the mouthpiece check valve 88 can block airflow 100 from the annular space 80 of the mouthpiece housing 70 to the port 74 of the mouthpiece housing. As shown in FIG. 2A, in the inspiration position, the mouthpiece check valve 88 can permit airflow 100 from the annular space 80 of the mouthpiece housing 70 to the port 74 of the mouthpiece housing. As shown in FIG. 2B, in the expiration position, the mouthpiece check valve 88 can direct airflow 100 from the annular space 80 of the mouthpiece housing 70 to the annular space 40 of the exhaust housing 30.

In further exemplary aspects, the mouthpiece housing 70 can comprise an internal flange 75 extending radially outwardly from the central opening 78 of the mouthpiece housing. In these aspects, the internal flange 75 can define an annular opening 77. In additional aspects, the mouthpiece check valve 88 can comprise an annular valve gasket that is configured for selective displacement relative to the annular opening 77 of the internal flange 75. In further aspects, when the mouthpiece check valve 88 is in the expiration position as shown in FIG. 2B, the annular valve gasket can engage the internal flange 75 to block airflow 100 through the annular opening 77 of the internal flange. In still further aspects, when the mouthpiece check valve 88 is in the inspiration position as shown in FIG. 2A, the annular valve gasket can be spaced from the internal flange 75 to permit airflow 100 between the annular space 80 of the mouthpiece housing 70 and the port 74 of the mouthpiece housing. In exemplary aspects, the annular valve gasket can be a low-pressure lift check valve that is shaped as a circular gasket.

In other exemplary aspects, and as shown in FIGS. 3A-3C, the exhaust housing 30 can be selectively rotatable among a plurality of rotational positions. In these aspects, each rotational position can correspond to a different inspiratory resistance to air flow between the annular spaces 40, 80 of the exhaust housing 30 and the mouthpiece housing 70. Optionally, in further aspects, the device 10 can be configured to produce an audible indication when the exhaust housing 30 reaches each respective rotational position of the plurality of rotational positions. In these aspects, it is contemplated that the exhaust housing 30 and the mouthpiece housing 70 can define respective structures that are configured to engage one another and then disengage from one another to produce an audible indication when the exhaust housing is rotated to a designated rotational position. Additionally, or alternatively, it is contemplated that the device can provide a visual indication to a user that a particular level of obstruction (resistance) has been achieved. Such visual indications can include, without limitation, a window defined in the exhaust housing that permits display of different (fixed) indicia within the exhaust housing rotates as disclosed herein.

In one exemplary device as shown in FIGS. 3A-3C, it is contemplated that the exhaust housing can be selectively rotated among three different rotational positions, with each rotational position representing a different threshold inspiratory pressure (required to permit inspiratory airflow), which is equal to the sum of the “cracking pressure threshold” of the exhaust check valve and the resistance provided by the fenestration 95 (i.e., the blocking component(s) 50 and the fenestrated layer 86). In the resting position (FIG. 1) or during expiration (FIG. 2B), the exhaust check-valve 46 can be flexed toward an exhaust tube, which leads to port 34. When sufficient inspiratory pressure is applied from the mouthpiece 90, the exhaust check valve 46 can flex to be seated on the internal stop 44. In its initial resting position (FIG. 3A), the fenestration 95 is large enough so that the threshold inspiratory pressure is 5 mm Hg (or another selected pressure value). The amount of inspiratory pressure required to continue to draw in air will depend upon the size of the fenestration. When the housing is rotated one ‘click’ to a second position (FIG. 3B), the total area of openings in the fenestration becomes smaller, contributing to more resistance to airflow so that the threshold inspiratory pressure increases to 10 mm Hg (or another selected pressure value that is greater than the pressure associated with the initial position). The exhaust housing 30 can then be rotated one more click to a third position (FIG. 3C), making the total area of openings in the fenestration even smaller so that the threshold inspiratory pressure increases to 15 mm Hg (or another selected pressure value that is greater than the first and second positions). Thus, in use, it is contemplated that the threshold inspiratory effort required to permit inspiration can remain consistent (standardized) at each respective position, and the amount of inspiratory effort required can be selectively adjusted as needed.

In further optional aspects, it is contemplated that at least two of the rotational positions can cause threshold inspiratory pressures of less than 5 mm Hg or less than 4 mm Hg. In still further optional aspects, it is contemplated that the threshold inspiratory pressures for all of the rotational positions (e.g., optionally, all three positions) can be less than 15 mm Hg, less than 10 mm Hg, less than 5 mm Hg, or less than 4 mm Hg. In various other aspects, a first threshold inspiratory pressure associated with a first rotational position can be between 2 and 10 mm Hg (optionally, between 2 and 5 mm Hg). A second threshold inspiratory pressure associated with a second rotational position can be greater than the first threshold inspiratory pressure and can be between about 4 mm Hg and 15 mm Hg (e.g., optionally, between 6 mm Hg and 8 mm Hg). A third threshold inspiratory pressure associated with a third rotational position can be greater than the second threshold inspiratory pressure and can be between about 7 mm Hg and 25 mm Hg (e.g., optionally, between 7 mm Hg and 10 mm Hg or between 10 mm Hg and 20 mm Hg).

In various optional aspects, the first position can cause a first threshold inspiratory pressure, and the second position can cause a second threshold inspiratory pressure that is between two times and four times the first threshold inspiratory pressure (e.g., optionally, about three times first threshold inspiratory pressure), thereby producing a relative pressure ratio of 2:1 to 4:1. In various optional aspects, the third position can cause a third threshold inspiratory pressure that is between four and six times the first threshold inspiratory pressure (e.g., optionally, about five times the first threshold inspiratory pressure), thereby producing a relative pressure ratio of 4:1 to 6:1. In further optional aspects, the third position can cause a threshold inspiratory pressure that is from six times the first threshold inspiratory pressure to ten times the first threshold inspiratory pressure (i.e., a pressure ratio ranging from 6:1 to 10:1). In other optional aspects, the second position can cause a threshold inspiratory pressure that is between two and six times the first threshold inspiratory pressure (i.e., a pressure ratio ranging from 2:1 to 6:1).

In further exemplary aspects, it is contemplated that the resistance to expiration remains consistent throughout use of the disclosed device. Additionally, or alternatively, it is contemplated that a third check valve can be incorporated into the mouthpiece assembly 60 proximate the port 74 (e.g., within a mouthpiece tube defining the port 74) to allow expired air to escape directly from the mouthpiece housing 70, thereby bypassing the fenestration during expiration. Thus, in some aspects, and in contrast to respiratory muscle training devices, it is contemplated that the disclosed devices can provide no resistance or substantially no resistance during expiration.

Devices Having an Exhaust Assembly with a Rotatable Airflow Adjustment Plate

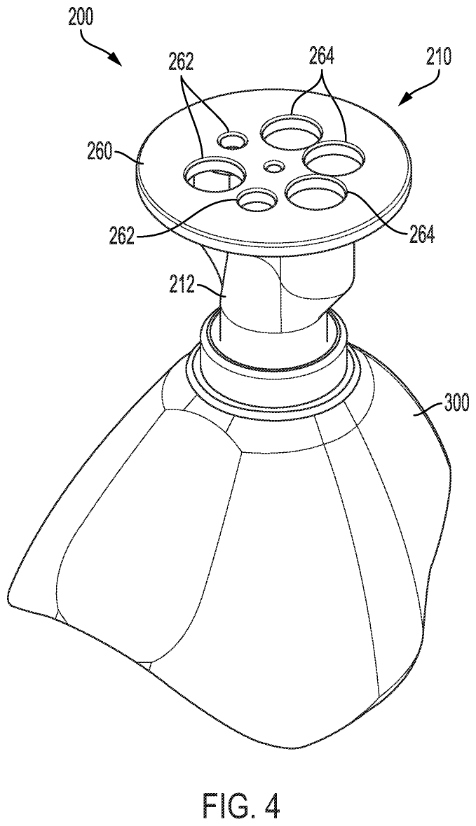

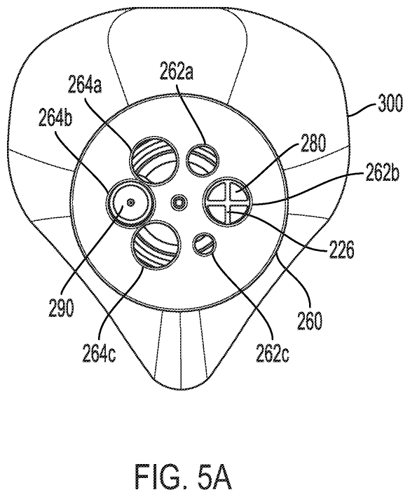

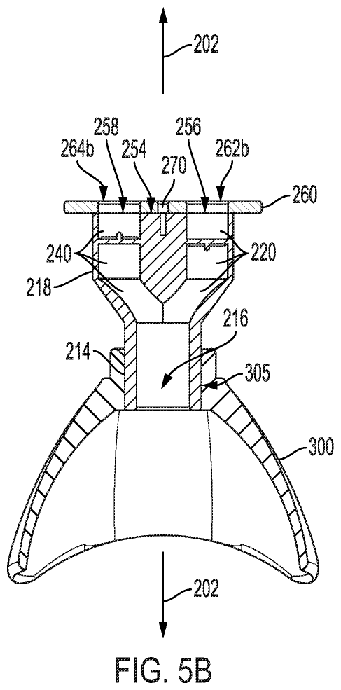

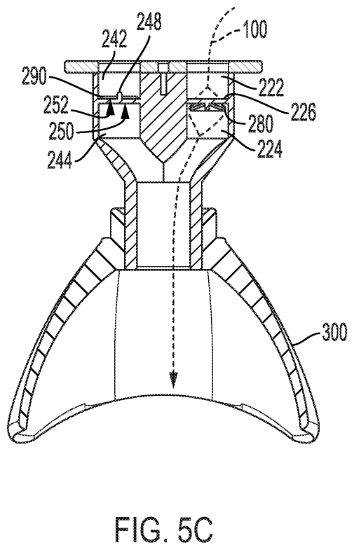

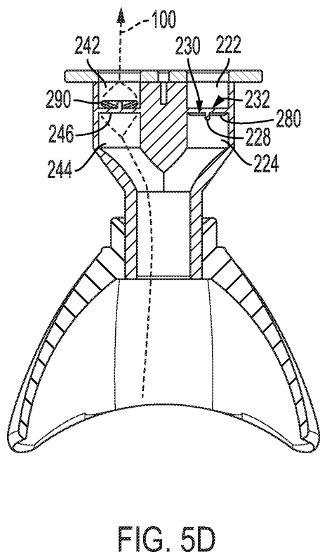

In alternative embodiments, and with reference to FIGS. 4-14, a device 200 for standardizing breathing efforts of a subject or patent can have a longitudinal axis 202 and comprise an exhaust assembly 210. The exhaust assembly 210 can have an exhaust housing 212 having a proximal portion 214 that defines a port 216 and an opposed distal portion 218 that defines respective inspiration and expiration pathways 220, 240. As shown in FIG. 5B, the distal portion 218 can have a distal end surface 254. In exemplary aspects, as shown in FIGS. 5B-5D, 6B-6D, and 7B-7D, the inspiration and expiration pathways 220, 240 can be in fluid communication with the port 216 and extend, respectively, to inlet and outlet openings 256 258 defined in the distal end surface 254.

In additional aspects, the exhaust assembly 210 can further comprise an airflow adjustment plate 260 that is rotatably coupled to the distal portion 218 of the exhaust housing 212. As shown, the airflow adjustment plate 260 can be configured to rotate about an axis that is parallel to or aligned with the longitudinal axis 202 of the device 200, which corresponds to the direction of breathing (exhale and inhale) of the subject. As shown in FIGS. 4, 5A, 6A, and 7A, the airflow adjustment plate 260 can define a first set of openings 262. In exemplary aspects, the first set of openings 262 of the airflow adjustment plate can comprise a plurality of openings having varying sizes. For example, as shown in the figures, it is contemplated that the first set of openings 262 can comprise first, second, and third openings 262a, 262b, 262c having varying sizes. As shown in FIGS. 5A-7D and further described below, rotation of the airflow adjustment plate 260 relative to the exhaust housing 212 among a plurality of rotational positions can increase or decrease resistance to air flow through the inlet opening 256 of the exhaust housing. More particularly, at each rotational position, a respective opening of the first set of openings 262 of the airflow adjustment plate 260 is positioned in alignment with the inlet opening 256 of the exhaust housing, thereby permitting adjustment of the resistance to air flow through the inlet opening of the exhaust housing.

As described above, it is contemplated that the airflow adjustment plate 260 can be selectively rotated among three different rotational positions, with each rotational position representing a different threshold inspiratory pressure (required to permit inspiratory airflow). In a first (e.g., starting position), it is contemplated that the first opening can be dimensioned so that the threshold inspiratory pressure is 5 mm Hg (or another selected pressure value). The amount of inspiratory pressure required to continue to draw in air will depend upon the size of the opening. When the plate 260 is rotated one position (e.g., one ‘click’) to a second position (e.g., corresponding to the second opening), the area of the opening becomes smaller, contributing to more resistance to airflow so that the threshold inspiratory pressure increases to 10 mm Hg (or another selected pressure value that is greater than the pressure associated with the initial position). The plate 260 can then be rotated one more click to a third position, (e.g., corresponding to the third opening), making the area of the opening even smaller so that the threshold inspiratory pressure increases to 15 mm Hg (or another selected pressure value that is greater than the first and second positions). Thus, in use, it is contemplated that the threshold inspiratory effort required to permit inspiration can remain consistent (standardized) at each respective position, and the amount of inspiratory effort required can be selectively adjusted as needed.

In further optional aspects, it is contemplated that at least two of the rotational positions can cause threshold inspiratory pressures of less than 5 mm Hg or less than 4 mm Hg. In still further optional aspects, it is contemplated that the threshold inspiratory pressures for all of the rotational positions (e.g., optionally, all three positions) can be less than 15 mm Hg, less than 10 mm Hg, less than 5 mm Hg, or less than 4 mm Hg. In various other aspects, a first threshold inspiratory pressure associated with a first rotational position can be between 2 and 10 mm Hg (optionally, between 2 and 5 mm Hg). A second threshold inspiratory pressure associated with a second rotational position can be greater than the first threshold inspiratory pressure and can be between about 4 mm Hg and 15 mm Hg (e.g., optionally, between 6 mm Hg and 8 mm Hg). A third threshold inspiratory pressure associated with a third rotational position can be greater than the second threshold inspiratory pressure and can be between about 7 mm Hg and 25 mm Hg (e.g., optionally, between 7 mm Hg and 10 mm Hg or between 10 mm Hg and 20 mm Hg).

In various optional aspects, the first position can cause a first threshold inspiratory pressure, and the second position can cause a second threshold inspiratory pressure that is between two times and four times the first threshold inspiratory pressure (e.g., optionally, about three times first threshold inspiratory pressure), thereby producing a relative pressure ratio of 2:1 to 4:1. In various optional aspects, the third position can cause a third threshold inspiratory pressure that is between four and six times the first threshold inspiratory pressure (e.g., optionally, about five times the first threshold inspiratory pressure), thereby producing a relative pressure ratio of 4:1 to 6:1. In further optional aspects, the third position can cause a threshold inspiratory pressure that is from six times the first threshold inspiratory pressure to ten times the first threshold inspiratory pressure (i.e., a pressure ratio ranging from 6:1 to 10:1). In other optional aspects, the second position can cause a threshold inspiratory pressure that is between two and six times the first threshold inspiratory pressure (i.e., a pressure ratio ranging from 2:1 to 6:1).

In further aspects, a first blocking component 280 (e.g., a first check valve) can be positioned within the inspiration pathway 220 of the exhaust housing 212, and a second blocking component 290 (e.g., a second check valve) can be positioned within the expiration pathway 240 of the exhaust housing. In response to inspiration through the inlet opening 256 and the inspiration pathway 220 of the exhaust housing 212 as shown in FIGS. 5C, 6C, and 7C, the first blocking component 280 can be configured to deform to permit airflow 100 to the port 216 of the exhaust housing 212, and the second blocking component 290 can be configured to prevent airflow through the expiration pathway 240. In response to expiration through the port 216 of the exhaust housing 212 and into the expiration pathway 240 as shown in FIGS. 5D, 6D, and 7D, the second blocking component 290 can be configured to deform to permit airflow 100 to the outlet opening 258 of the exhaust housing 212, and the first blocking component 280 can be configured to restrict or prevent airflow through the inspiration pathway 220.

In further exemplary aspects, and as shown in FIGS. 5B-5D, 6B-6D, and 7B-7D, the inspiration pathway 220 can comprise a first compartment 222 in fluid communication with the inlet opening 256 of the exhaust housing 212 and a second compartment 224 spaced proximally from the first compartment and positioned in longitudinal alignment with the first compartment of the inspiration pathway. As shown, the second compartment 224 can be in fluid communication with the port 216 of the exhaust housing 212. In these aspects, the inspiration pathway 220 can further comprise a support frame 226 positioned between the first and second compartments 222, 224. In use, the support frame 226 can permit airflow through the inspiration pathway when the blocking component 280 is in an inspiration position (i.e., deformed to permit airflow around at least portions of the periphery of the blocking component). For example, it is contemplated that that the support frame 226 can define at least one opening (optionally, a plurality of openings) that permit airflow through the frame. An exemplary top view of the support frame 226 is provided in FIG. 5A, showing a plurality of support arms that intersect at a center portion of the support frame to define four openings. In further aspects, the support frame 226 can comprise a projection 228 that extends proximally within the second compartment 224. Optionally, the projection 228 can extend from the center portion of the support frame. In further exemplary aspects, the first blocking component 280 can be secured to the projection 228. Optionally, it is contemplated that the first blocking component 280 can define a central opening that receives a portion of the projection 228, with the interior surfaces of the first blocking component 280 that define the central opening being adhesively, mechanically, and/or frictionally secured to the outer surface of the projection 228. Optionally, it is contemplated that the blocking component 280 can be secured to the projection 228 using a form-fit. However, other securing mechanisms or approaches can be used.

In additional aspects, and as shown in FIGS. 5B-5D, 6B-6D, and 7B-7D, the expiration pathway 240 can comprise a first compartment 242 in fluid communication with the outlet opening 258 of the exhaust housing 212 and a second compartment 244 spaced proximally from the first compartment of the expiration pathway and positioned in longitudinal alignment with the first compartment of the expiration pathway. As shown, the second compartment 244 can be in fluid communication with the port 216 of the exhaust housing 212. In these aspects, the expiration pathway 240 can further comprise a support frame 246 positioned between the first and second compartments 242, 244 of the expiration pathway. In use, the support frame 246 can permit airflow through the expiration pathway 240 when the blocking component 290 is in an expiration position (i.e., deformed to permit airflow around at least portions of the periphery of the blocking component). For example, it is contemplated that that the support frame 246 can define at least one opening (optionally, a plurality of openings) that permit airflow through the frame. Optionally, the support frame 246 can have the same structure as support frame 226 (see FIG. 5A, for example). In further aspects, the support frame 246 can comprise a projection 248 that extends distally within the first compartment 242 of the expiration pathway 240. Optionally, the projection 248 can extend from a center portion of the support frame 246. In further exemplary aspects, the second blocking component 290 can be secured to the projection 248. Optionally, it is contemplated that the second blocking component 290 can define a central opening that receives a portion of the projection 248, with the interior surfaces of the second blocking component 290 that define the central opening being adhesively, mechanically, and/or frictionally secured to the outer surface of the projection 248. Optionally, it is contemplated that the blocking component 290 can be secured to the projection 248 using a form-fit. However, other securing mechanisms or approaches can be used.

As shown in FIGS. 5C, 6C, and 7C, in response to inspiration through the inlet opening 256 and the inspiration pathway 220 of the exhaust housing 212, the first blocking component 280 can be configured to deform proximally within the second compartment 224 of the inspiration pathway 220 to permit airflow 100 through the support frame 226 of the inspiration pathway. As shown in FIGS. 5D, 6D, and 7D, in response to expiration through the port 216 of the exhaust housing 212, the second blocking component 290 can be configured to deform distally within the first compartment 242 of the expiration pathway 240 to permit airflow 100 through the support frame 246 of the expiration pathway.

In exemplary aspects, as shown in FIG. 5D, the support frame 226 of the inspiration pathway 220 can have opposing proximal and distal surfaces 230, 232. In these aspects, the projection 228 of the support frame 226 of the inspiration pathway 220 can extend proximally from the proximal surface 230, and the first blocking component 280 can start in (optionally, be biased toward) a resting position (as shown in FIGS. 5B, 5D, 6B, 6D, 7B, and 7D) in which the first blocking component 280 abuts at least a portion of the proximal surface 230 of the support frame 226 of the inspiration pathway 220 and sufficiently overlies or covers the support frame (i.e., sufficiently covers the openings of the support frame) to prevent airflow from the first compartment 222 of the inspiration pathway 220 to the second compartment 224 of the inspiration pathway.

In still further exemplary aspects, as shown in FIG. 5C, the support frame 246 of the expiration pathway 240 can have opposing proximal and distal surfaces 250, 252. In these aspects, the projection 248 of the support frame 246 of the expiration pathway 240 can extend distally from the distal surface 252, and the second blocking component 290 can be biased toward a resting position (as shown in FIGS. 5B-5C, 6B-6C, and 7B-7C) in which the second blocking component 290 abuts at least a portion of the distal surface 252 of the support frame 246 of the expiration pathway 240 and sufficiently overlies or covers the support frame 246 (i.e., sufficiently covers the openings of the support frame) to prevent airflow from the second compartment 244 of the expiration pathway 240 to the first compartment 242 of the expiration pathway.

Optionally, in some aspects, at least one of the first and second blocking components 280, 290 can comprise silicone discs. Optionally, both blocking components 280, 290 can comprise silicone discs. Although disclosed herein as discs, it is contemplated that the blocking components 280, 290 can have any desired cross-sectional profile, including a round, oval, elliptical, triangular, rectangular, or complex profile.

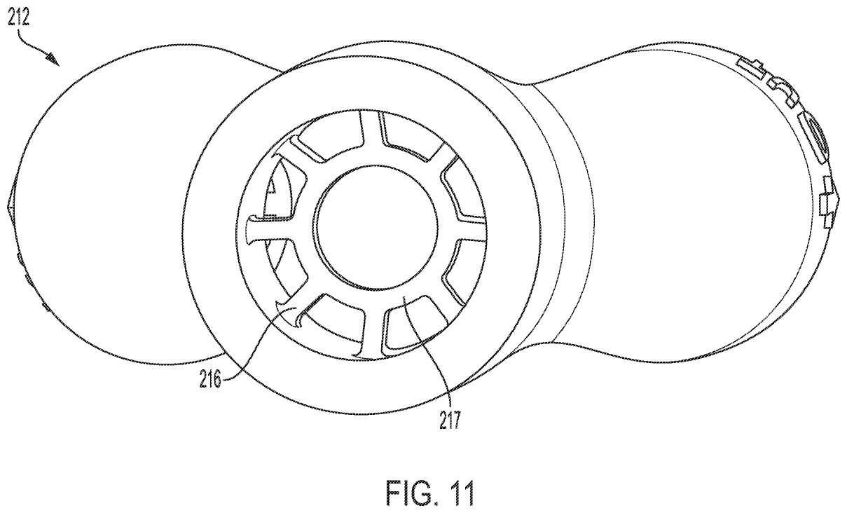

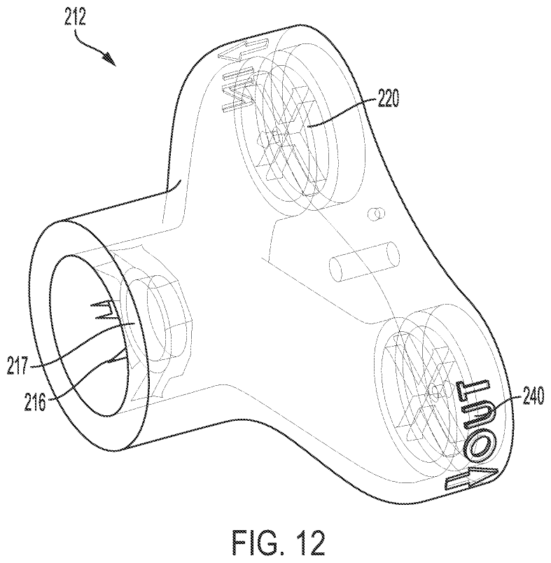

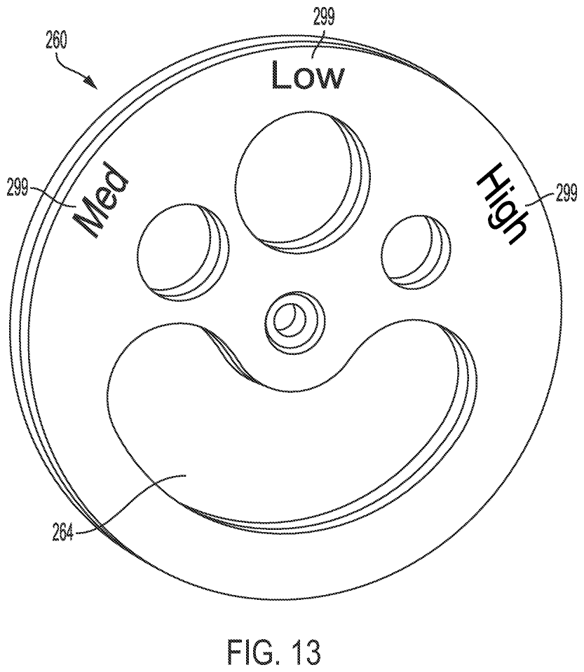

In further exemplary aspects, and as shown in FIGS. 11-12, the device 200 can further comprise a grate 217 that is positioned within the exhaust housing 212, between the blocking components 280, 290 and the port 216. In use, it is contemplated that the grate can have a structure that extends across the opening within the exhaust housing 212 and defines sufficient openings to having minimal impact on the flow of air through the exhaust housing. In use, the grate 217 can be configured to block the passage of any blocking components 280, 290 that may become unattached from their mounting/coupling points during use of the device, thereby preventing inhalation of the blocking components by the subject.

Optionally, in some aspects, the first and second blocking components 280, 290 can have respective thicknesses of less than 0.025 inches.

In exemplary aspects, and as shown in FIGS. 5B-5D, 6B-6D, and 7B-7D, the airflow adjustment plate 260 can be rotatably coupled to the exhaust housing 212 using a fastener 270. Optionally, in these aspects, the fastener can comprise a screw, a pin, a bolt, or a post that permits free rotation of the airflow adjustment plate 260 while securely coupling the airflow adjustment plate to the exhaust housing 212.

In further exemplary aspects, the airflow adjustment plate 260 comprises plastic.

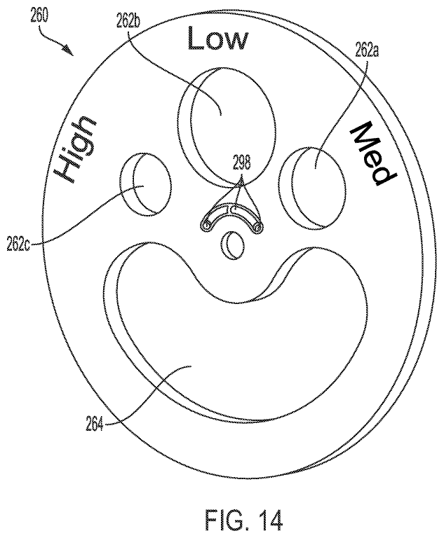

Optionally, in exemplary aspects, the airflow adjustment plate 260 can further define a second set of openings 264 (in addition to the first set of openings 262). In these aspects, and as shown in FIGS. 4, 5A, 6A, and 7A, the first and second sets of openings 262, 264 of the airflow adjustment plate 260 can be spaced along respective arcuate paths (measured through the center points of each opening in the set). In these aspects, at each rotational position, a respective opening 264 of the second set of openings of the airflow adjustment plate 260 can be positioned in alignment with the outlet opening 258 of the exhaust housing 212. Optionally, in exemplary aspects, the second set of openings 264 of the airflow adjustment plate can comprise a plurality of openings having equal diameters or sizes. For example, as shown in the figures, it is contemplated that the second set of openings 264 can comprise first, second, and third openings 264a, 264b, 264c having equal diameters and sizes. Thus, while the airflow resistance for the inspiration pathway can be adjusted by rotating the airflow adjustment plate to vary the size of the first opening 262 that is in alignment with the inlet opening 256 of the exhaust housing 212, the amount of airflow resistance at the outlet opening 258 of the exhaust housing (to the extent there is any) can remain substantially consistent, with the size of the second opening 264 remaining the same regardless of the rotational position. In still further aspects, and with reference to FIGS. 13 and 14, the second set of openings can comprise a single opening. More particularly, it is contemplated that the first, second, and third openings of the second set of openings can be combined into a single opening 264 that is positioned over the outlet opening 258 when each of the first openings 262a,b,c is positioned over the inlet opening 256. For example, the single opening 264 can have an arcuate length and a constant radial dimension (width) along its arcuate length. In this way, in each position, the single opening 264 can expose the same area of the outlet opening 258, corresponding to an equal pressure drop thereacross. Optionally, during expiration, it is contemplated that the second opening(s) 264 can be configured to provide no added airflow resistance or substantially no added airflow resistance.

In still further exemplary aspects, and as shown in FIG. 4, the airflow adjustment plate 260 can have a diameter that exceeds a maximum diameter of the exhaust housing 212.

In still further exemplary aspects, the arcuate paths of the first and second sets of openings have differing radii of curvature. For example, as shown in FIG. 5A, the arcuate path of the second set of openings 264a, 264b, 264c has a greater radius of curvature than the arcuate path of the first set of openings 262a, 262b, 262c. It is contemplated that the variation in the arcuate paths of the two sets of openings can ensure that at each rotational position, an opening of the first set of openings 262 is aligned with the inlet opening 256 at the same time an opening of the second set of openings 264 is aligned with the outlet opening 258.

In still further exemplary aspects, the first and second sets of openings of the airflow adjustment plate each can comprise from 2 to 5 openings. For example, in some aspects, both the first and second sets of openings can comprise three openings. Optionally, in these aspects, as shown in FIGS. 5A, 6A, and 7A, a first opening 262b of the first set of openings has an area corresponding to about 100% of an area of the inlet opening 256 of the exhaust housing 212, a second opening 262a of the first set of openings has an area corresponding to about 50% of the area of the inlet opening of the exhaust housing, and a third opening 262c of the first set of openings has an area corresponding to about 25% of the area of the inlet opening of the exhaust housing.

Optionally, at least one opening of the first set of openings 262 of the airflow adjustment plate 260 can have a diameter that is less than the diameter of each opening of the second set of openings 264 of the airflow adjustment plate. In exemplary aspects, it is contemplated that each opening of the first set of openings 262 of the airflow adjustment plate 260 can have a diameter that is less than the diameter of each opening of the second set of openings 264 of the airflow adjustment plate.

Optionally, each opening of the first set of openings 262 of the airflow adjustment plate 260 can have a different diameter than each other opening of the first set of openings.

Optionally, each opening of the first set of openings 262 of the airflow adjustment plate 260 can have a different area than each other opening of the first set of openings.

By providing first and second sets of openings 262, 264 on a single adjustment plate 260 as disclosed herein, it is contemplated that resistance to both input and output airflow can be regulated by rotating the adjustment plate 260. Thus, in exemplary aspects, it is contemplated that the device 200 can consist of a single adjustment plate (or other rotatable component) that adjusts airflow through the device. In further aspects, it is contemplated that the adjustment plate 260 can be oriented perpendicular or substantially perpendicular to the longitudinal axis 202 of the device (and thus, perpendicular or substantially perpendicular to the direction of breathing by the patient). Thus, in these aspects, the first and second sets of openings 262, 264 can be co-planar within a plane that is perpendicular or substantially perpendicular to the longitudinal axis 202 of the device (and thus, perpendicular or substantially perpendicular to the direction of breathing by the patient).

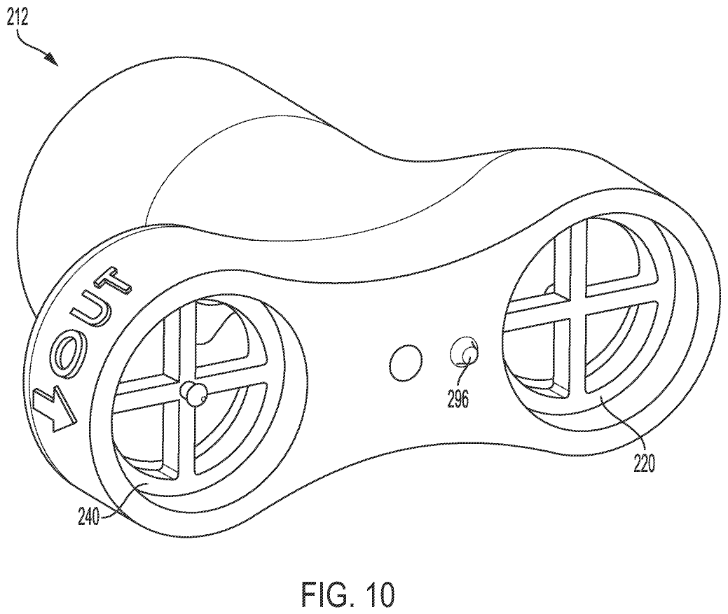

In further exemplary aspects, at least one of the airflow adjustment plate 260 or the exhaust housing 212 can be configured to provide a tactile or audible indication in response to movement among the plurality of rotational positions of the airflow adjustment plate. In these aspects, it is contemplated that the exhaust housing 212 and the airflow adjustment plate 260 can define respective structures that are configured to engage one another and then disengage from one another to produce an audible and/or tactile indication when the airflow adjustment plate 260 is rotated to a designated rotational position. For example, referring to FIGS. 10 and 14, optionally, the exhaust housing 212 can define a projection 296 that can be received into a corresponding notch 298 defined in the airflow adjustment plate 260 when one of the first, second, or third openings 262a,b,c is concentrically aligned with the inlet opening 256, thereby indicating alignment and retaining the adjustment plate 260 in position. Still further, adjustment plate 260 can have indicators 299 indicating the amount of resistance for the corresponding opening 262 (for example, a textual indicator such as “High,” “Low,” or “Med”/“Medium”). The indicators 299 can be positioned on the front of the airflow adjustment plate 260 for viewing by a medical professional viewing the front of the device 200. Thus, when the device is used while obtaining an echocardiogram (or ultrasound) of a subject, the indicators can face away from the face of the patient and be readily viewable by the medical professional (e.g., an electrograph technician) so that the medical professional can easily determine the inspiratory resistance level (pressure) and be able to evaluate RAP based on monitoring of the diameter of the inferior vena cava of the subject.

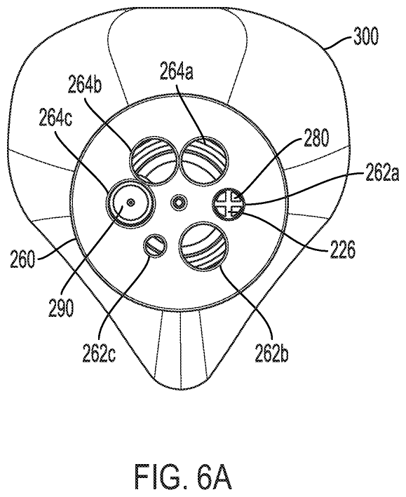

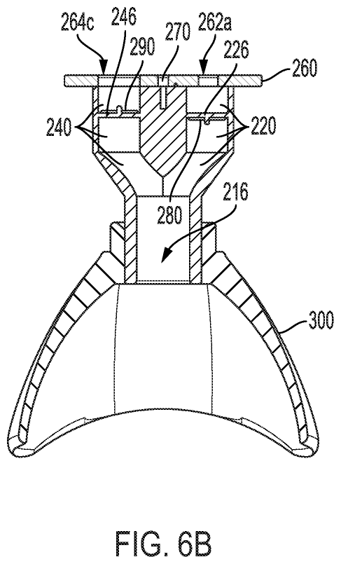

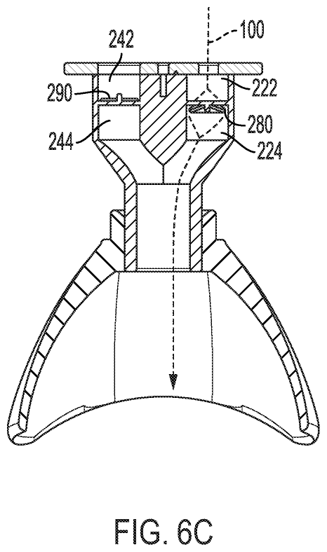

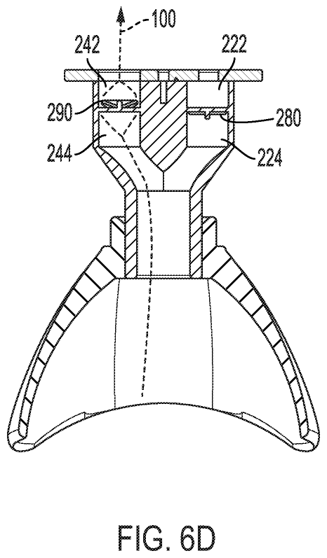

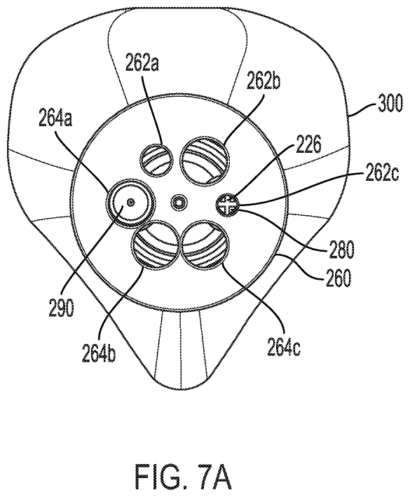

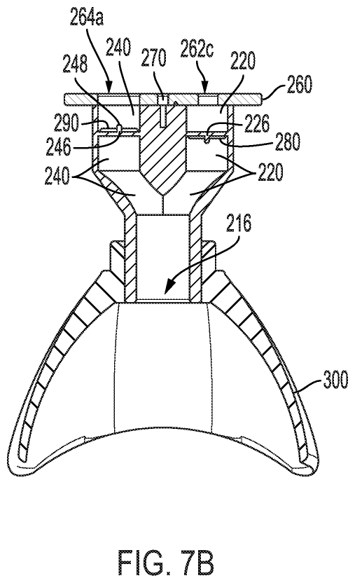

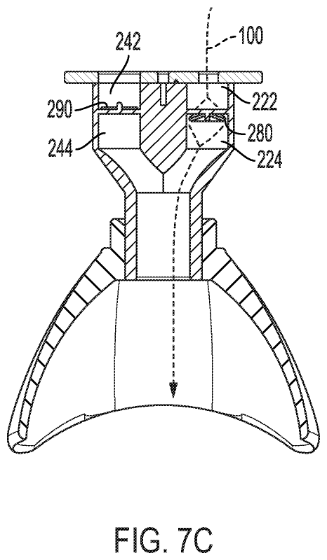

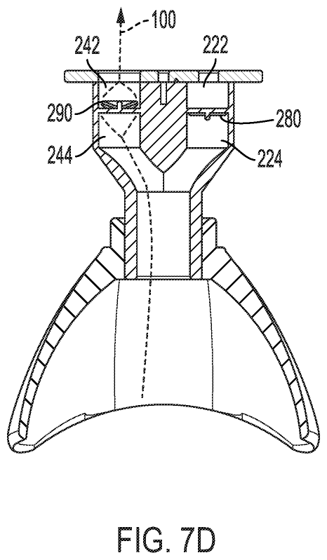

In use, as shown in FIGS. 5A-5D, the airflow adjustment plate 260 can initially start in a first rotational position. In this example position, the first opening 262b can correspond to a minimum amount of resistance to airflow through the inspiration pathway 220. Optionally, as shown, opening 262b can be provided at an intermediate position along the arcuate path of the first set of openings. As shown in FIGS. 6A-7D, if a clinician or the subject wishes to provide an increase in airflow resistance, the airflow adjustment plate 260 can be rotated in a first direction to provide an intermediate level of resistance (FIGS. 6A-6D) or in an opposing second direction to provide an increased level of resistance (FIGS. 7A-7D).

As shown in FIGS. 5B, 6B, and 7B, prior to the start of controlled breathing by the subject, the first and second blocking components 280, 290 can be in a resting position. As shown in FIGS. 5C, 6C, and 7C, when inspiration occurs, the negative pressure applied by the subject can deform the first blocking component 280 in a proximal direction to reduce the operative circumference of the blocking component and permit airflow 100 between the first compartment 222 and the second compartment 224. During inspiration, the support frame 246 prevents the second blocking component 290 from deforming in a proximal direction, thereby preventing airflow through the expiration pathway 240. As shown in FIGS. 5D, 6D, and 7D, during expiration, the positive pressure applied by the subject can deform the second blocking component 290 in a distal direction to reduce the operative circumference of the blocking component and permit airflow 100 between the second compartment 244 and the first compartment 242. During expiration, the support frame 226 prevents the first blocking component 280 from deforming in a proximal direction, thereby preventing airflow through the inspiration pathway 220.

In still further exemplary aspects, and as shown in FIGS. 4-7D, the device 200 can further comprise a patient interface 300 that is configured for engagement with the port 216 of the exhaust housing 212 and placement over or within the nose and/or face of a subject. In these aspects, as shown in FIG. 5B, the patient interface 300 can define a port opening 305 that is configured to receive the port 216 of the exhaust housing 212 such that the exhaust housing frictionally engages the patient interface. Alternatively, it is contemplated that the patient interface 300 can define a port opening 305 that is configured for receipt within the port 216 of the exhaust housing 212 such that the exhaust housing frictionally engages the patient interface. Optionally, the patient interface 300 can comprise a strap (not shown) that can be used to secure the patient interface to the head of the subject. Alternatively, the strap can be provided separately from the patient interface 300 and then independently placed over the patient interface to selectively secure the patient interface to the head of the subject.

Optionally, as shown in FIGS. 4-7D, the patient interface 300 can be provided in the form of a mask. However, in other aspects, it is contemplated that the patient interface 300 can be provided in the form of a mouthpiece (not shown) that is configured for engagement with the port 216 of the exhaust housing 212. In these aspects, the mouthpiece can be received within or receive the port 216 of the exhaust housing, thereby providing fluid communication between the port 216 of the exhaust housing and a center channel of the mouthpiece, which is in communication with the mouth of the subject. Optionally, it is contemplated that the mouthpiece can be a standard medical mouthpiece that is provided separately from the device 200. Additionally, or alternatively, it is contemplated that the mouthpiece can be configured (optionally, customized) for a precise fit with the exhaust housing. It is further contemplated that the device 200 can further comprise a nose clip for restricting nasal breathing while a subject provides controlled breathing through the patient interface (e.g., mouthpiece).

In exemplary aspects, a kit can comprise an exhaust housing as disclosed herein along with a plurality of airflow adjustment plates configured to be rotatably coupled to the distal portion of the exhaust housing. In these aspects, the first set of openings of each airflow adjustment plate can differ from the first set of openings of each other airflow adjustment plate in diameter, number, or area, thereby permitting substitution of the airflow adjustment plates for one another to provide further modification of the resistance to airflow through the inspiration pathway 220. The kits can further comprise other components of the disclosed device 200, including the first and second blocking components 280, 290, and a fastener for removably and rotatably coupling the airflow adjustment plates 260 to the exhaust housing 212.

### Methods of Standardizing Breathing Effort

In exemplary applications, methods of using the disclosed devices can produce standardized breathing effort in a subject or patient. The disclosed methods can further provide a standardized breathing pattern in a subject or patient. As further described above, and in contrast to other impedance threshold devices, the threshold respiratory effort of the disclosed devices can be increased or decreased as needed. In use of the devices disclosed herein, it is contemplated that the expiration of air can be unrestricted, with resistance being increased during inspiration but not during expiration.

In exemplary aspects, with respect to the embodiments of the device depicted in FIGS. 1-3C, a method can comprise operatively positioning the mouthpiece of the device relative to a subject. In these aspects, the method can further comprise using the device to deliver inspiratory air to the subject in response to inspiration of the subject that exceeds a cracking pressure threshold of the exhaust check valve of the exhaust assembly. In response to exceeding the cracking pressure threshold, the exhaust check valve can flex from a resting position to an inspiration position that permits airflow from the port of the exhaust housing to the annular space of the exhaust housing. In response to the subject exceeding the cracking pressure threshold and airflow from the port of the exhaust housing to the annular space of the exhaust housing, the mouthpiece check valve of the mouthpiece assembly can be displaced from a resting position to an inspiration position as disclosed herein. In their respective inspiration positions, the exhaust check valve and the mouthpiece check valve can cooperate to determine an inspiration flow pathway within the device. In the inspiration position, the exhaust check valve can block airflow through the central opening of the mouthpiece housing and direct airflow from the port of the exhaust housing to the annular space of the exhaust housing. The mouthpiece check valve can permit airflow from the annular space of the mouthpiece housing to the port of the mouthpiece housing.

In further aspects, with respect to the embodiments of the device depicted in FIGS. 1-3C, the method can comprise using the device to receive expired air from the subject. In response to expiration by the subject, the mouthpiece check valve and the exhaust check valve can move from their respective inspiration positions to respective expiration positions. In their respective expiration positions, the mouthpiece check valve and the exhaust check valve can cooperate to determine an expiration flow pathway within the device. In the expiration position, the exhaust check valve can permit airflow through the central opening of the exhaust housing and direct airflow from the central opening of the exhaust housing to the annular spaces of the mouthpiece and exhaust housings, and the mouthpiece check valve can block airflow from the annular space of the mouthpiece housing to the port of the mouthpiece housing and direct airflow from the annular space of the mouthpiece housing to the annular space of the exhaust housing.

Optionally, in still further aspects, and with respect to the embodiments of the device depicted in FIGS. 1-3C, the method can comprise rotating the exhaust housing relative to the mouthpiece housing to selectively adjust a threshold respiratory effort of the device.

In exemplary aspects, with respect to the embodiments of the device depicted in FIGS. 4-7D, a method can comprise operatively positioning the port of the exhaust housing of the device relative to a subject. In these aspects, the method can further comprise using the device to deliver inspiratory air to the subject in response to inspiration of the subject that exceeds a cracking pressure threshold of a first blocking component positioned within the inspiration pathway of the exhaust assembly. In response to exceeding the cracking pressure threshold, the first blocking component can flex from a resting position to an inspiration position that permits airflow from the inlet opening of the exhaust housing to the port of the exhaust housing.

In further aspects, the method can comprise using the device to receive expired air from the subject. In response to expiration by the subject, the first blocking component can return to the resting position, and a second blocking component positioned within the expiration pathway of the exhaust assembly can flex from a resting position to an expiration position that permits airflow from the port of the exhaust housing to the outlet opening of the exhaust housing (such that the expiration of air is unrestricted, with no increase in resistance).

In additional aspects, the method can further comprise rotating the airflow adjustment plate to selectively adjust a threshold respiratory effort of the device.

Optionally, in further aspects, the method can further comprise capturing an echocardiogram or ultrasound of the subject during use of the device. Optionally, in exemplary aspects, the settings during the use of the device can be associated with the echocardiogram or ultrasound. For example, in these aspects, the echocardiogram or ultrasound report (digital or hard copy) can include an indication of the resistance setting (or settings) that was used during capture of the echocardiogram or ultrasound. It is contemplated that the electrocardiogram/ultrasound machine used to capture the echocardiogram or ultrasound can provide a user interface that allows for inputting the resistance settings that are used during echocardiogram or ultrasound capture. It is further contemplated that the processing unit(s) of the electrocardiogram/ultrasound machine can associate the resistance settings with the recorded echocardiogram or ultrasound and include the inputted resistance settings on a display screen or printable report that includes the echocardiogram or ultrasound information. Additionally, or alternatively, it is contemplated that the impedance threshold device can include a digital encoder or other sensor that is configured to sense a rotational position of the resistance structures disclosed herein, with the sensed rotational position being indicative of a particular resistance level. It is further contemplated that the sensor can be communicatively coupled to a wireless transmitter (optionally, through an onboard microcontroller). The wireless transmitter can be communicatively coupled to a wireless receiver of the electrocardiogram/ultrasound machine such that information about the rotational position (and resistance) of the device can be communicated to the ultrasound machine. The wireless receiver of the ultrasound machine can be communicatively coupled to the processing unit(s) of the ultrasound machine to allow for inclusion of the resistance information on the echocardiogram or ultrasound reports or displays.

As further disclosed herein, a medical professional can determine RAP by observing how the diameter of the inferior vena cava (the large vein the returns blood to the heart from the abdomen, pelvis, and legs) changes with breathing. In exemplary aspects, it is contemplated that the processing unit(s) of the electrocardiogram/ultrasound machine can be configured to execute software that determines the diameter of the inferior vena cava and, optionally, associates the determined diameter of the inferior vena cava with the resistance level provided by the device.

### Exemplary Aspects

In view of the described devices, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A device comprising: an exhaust assembly having: an exhaust housing having a distal end that defines a port and an opposed proximal end that defines a central opening and an annular space positioned radially between the central opening and an outer surface of the exhaust housing; and at least one selectively expandable and contractible blocking component received within the annular space of the exhaust housing and coupled to the exhaust housing; a mouthpiece assembly having: a mouthpiece housing having a proximal end that defines a port and an opposed distal end that defines a central opening and an annular space positioned radially between the central opening and an outer surface of the mouthpiece housing; and a mouthpiece positioned in fluid communication with the port of the mouthpiece housing, wherein the proximal end of the exhaust housing is rotatably coupled to the distal end of the mouthpiece housing, and wherein rotation of the exhaust housing relative to the mouthpiece housing selectively expands or contracts the at least one selectively expandable and contractible blocking component of the exhaust assembly to increase or decrease resistance to air flow between the annular spaces of the mouthpiece housing and the exhaust housing.

Aspect 2: The device of aspect 1, wherein the mouthpiece assembly further comprises a fenestrated layer extending circumferentially within the annular space of the mouthpiece housing, wherein expansion or contraction of the at least one selectively expandable and contractible blocking component of the exhaust assembly increases or decreases resistance to air flow through the fenestrated layer of the mouthpiece housing.

Aspect 3: The device of aspect 1 or aspect 2, wherein the blocking component comprises a plurality of fan elements.

Aspect 4: The device of any one of the preceding aspects, wherein the exhaust assembly further comprises an exhaust check valve configured for movement about and between an inspiration position and an expiration position, wherein in the inspiration position, the exhaust check valve blocks airflow through the central opening of the mouthpiece housing and directs airflow from the port of the exhaust housing to the annular space of the exhaust housing, and wherein in the expiration position, the exhaust check valve permits airflow through the central opening of the exhaust housing.

Aspect 5: The device of aspect 4, wherein in the expiration position, the exhaust check valve directs airflow from the central opening of the exhaust housing to the annular spaces of the mouthpiece and exhaust housings.

Aspect 6: The device of aspect 4 or aspect 5, wherein the exhaust check valve comprises a diaphragm.

Aspect 7: The device of any one of aspecst 4-6, wherein the proximal end of the exhaust housing has an annular wall that defines the central opening of the exhaust housing, and wherein the annular wall defines a seat configured to support the exhaust check valve when the exhaust check valve is in the inspiration and expiration positions.

Aspect 8: The device of aspect 7, wherein the distal end of the mouthpiece housing has an annular wall at least partially received within the central opening of the exhaust housing, wherein, when the exhaust check valve is in the inspiration position, the annular wall of the mouthpiece housing defines a stop surface that is configured to contact the exhaust check valve to block airflow through the central opening of the mouthpiece assembly.

Aspect 9: The device of aspect 8, wherein, when the exhaust check valve is in the expiration position, the stop surface of the annular wall of the mouthpiece housing is spaced from the exhaust check valve to permit airflow from the central opening of the mouthpiece housing into the central opening of the exhaust housing.

Aspect 10: The device of any one of the preceding aspects, wherein the mouthpiece assembly further comprises a mouthpiece check valve configured for movement about and between an inspiration position and an expiration position, wherein in the expiration position, the mouthpiece check valve blocks airflow from the annular space of the mouthpiece housing to the port of the mouthpiece housing, and wherein in the inspiration position, the mouthpiece check valve permits airflow from the annular space of the mouthpiece housing to the port of the mouthpiece housing.

Aspect 11: The device of aspect 10, wherein in the expiration position, the mouthpiece check valve directs airflow from the annular space of the mouthpiece housing to the annular space of the exhaust housing.

Aspect 12: The device of aspect 10 or aspect 11, wherein the mouthpiece housing comprises an internal flange extending radially outwardly from the central opening of the mouthpiece housing, wherein the internal flange defines an annular opening, and wherein the mouthpiece check valve comprises an annular valve gasket that is configured for selective displacement relative to the annular opening of the internal flange.

Aspect 13: The device of aspect 12, wherein when the mouthpiece check valve is in the expiration position, the annular valve gasket engages the internal flange to block airflow through the annular opening of the internal flange.

Aspect 14: The device of aspect 12 or aspect 13, wherein when the mouthpiece check valve is in the inspiration position, the annular valve gasket is spaced from the internal flange to permit airflow between the annular space of the mouthpiece housing and the port of the mouthpiece housing.

Aspect 15: The device of any one of the preceding aspects, wherein the exhaust housing is selectively rotatable among a plurality of rotational positions, wherein each rotational position corresponds to a different inspiratory resistance to air flow between the annular spaces of the exhaust housing and the mouthpiece housing.

Aspect 16: The device of aspect 15, wherein the device is configured to produce an audible indication when the exhaust housing reaches each respective rotational position of the plurality of rotational positions.

Aspect 17: A method of using the device of any one of the preceding claims.

Aspect 18: The method of aspect 17, comprising: operatively positioning the mouthpiece of the device relative to a subject; and using the device to deliver inspiratory air to the subject in response to inspiration of the subject that exceeds a cracking pressure threshold of an exhaust check valve of the exhaust assembly, wherein in response to exceeding the cracking pressure threshold, the exhaust check valve flexes from a resting position to an inspiration position that permits airflow from the port of the exhaust housing to the annular space of the exhaust housing.

Aspect 19: The method of aspect 18, wherein in response to the subject exceeding the cracking pressure threshold and airflow from the port of the exhaust housing to the annular space of the exhaust housing, a mouthpiece check valve of the mouthpiece assembly is displaced from a resting position to an inspiration position, wherein in their respective inspiration positions, the exhaust check valve and the mouthpiece check valve cooperate to determine an inspiration flow pathway within the device.

Aspect 20: The method of aspect 19, wherein in the inspiration position, the exhaust check valve blocks airflow through the central opening of the mouthpiece housing and directs airflow from the port of the exhaust housing to the annular space of the exhaust housing, and the mouthpiece check valve permits airflow from the annular space of the mouthpiece housing to the port of the mouthpiece housing.

Aspect 21: The method of aspect 19 or aspect 20, further comprising: using the device to receive expired air from the subject, wherein in response to expiration by the subject, the mouthpiece check valve and the exhaust check valve move from their respective inspiration positions to respective expiration positions, wherein in their respective expiration positions, the mouthpiece check valve and the exhaust check valve cooperate to determine an expiration flow pathway within the device.

Aspect 22: The method of aspect 21, wherein in the expiration position, the exhaust check valve permits airflow through the central opening of the exhaust housing and directs airflow from the central opening of the exhaust housing to the annular spaces of the mouthpiece and exhaust housings, and the mouthpiece check valve blocks airflow from the annular space of the mouthpiece housing to the port of the mouthpiece housing and directs airflow from the annular space of the mouthpiece housing to the annular space of the exhaust housing.

Aspect 23: The method of any one of aspecst 17-22, further comprising rotating the exhaust housing relative to the mouthpiece housing to selectively adjust a threshold respiratory effort of the device.

Aspect 24: The method of any one of aspects 17-23, further comprising capturing an echocardiogram of the subject during use of the device.

Aspect 24A: The method of aspect 24, further comprising using the echocardiogram to determine an estimate of right atrium pressure (RAP) of the subject.

Aspect 25: A device having a longitudinal axis and comprising: an exhaust assembly having: an exhaust housing having a proximal portion that defines a port and an opposed distal portion that defines respective inspiration and expiration pathways, wherein the distal portion has a distal end surface, and wherein the inspiration and expiration pathways are in fluid communication with the port and extend, respectively, to inlet and outlet openings defined in the distal end surface; and an airflow adjustment plate rotatably coupled to the distal portion of the exhaust housing, wherein the airflow adjustment plate defines a first set of openings, wherein the first set of openings of the airflow adjustment plate comprises a plurality of openings having varying sizes, and wherein rotation of the airflow adjustment plate relative to the exhaust housing among a plurality of rotational positions increases or decreases resistance to air flow through the inlet opening of the exhaust housing, wherein at each rotational position, a respective opening of the first set of openings of the airflow adjustment plate is positioned in alignment with the inlet opening of the exhaust housing.

Aspect 26: The device of aspect 25, further comprising: a first blocking component positioned within the inspiration pathway of the exhaust housing; and a second blocking component positioned within the expiration pathway of the exhaust housing, wherein, in response to inspiration through the inlet opening and the inspiration pathway of the exhaust housing, the first blocking component is configured to deform to permit increased airflow to the port of the exhaust housing, and the second blocking component is configured to prevent airflow through the expiration pathway, and wherein, in response to expiration through the port of the exhaust housing, the second blocking component is configured to deform to permit airflow to the outlet opening of the exhaust housing, and the first blocking component is configured to restrict airflow through the inspiration pathway.

Aspect 27: The device of aspect 26, wherein the inspiration pathway comprises: a first compartment in fluid communication with the inlet opening of the exhaust housing; a second compartment spaced proximally from the first compartment and positioned in alignment with the first compartment; and a support frame positioned between the first and second compartments, wherein the support frame permits airflow through the inspiration pathway and comprises a projection that extends proximally within the second compartment, wherein the first blocking component is secured to the projection.

Aspect 28: The device of aspect 27, wherein the expiration pathway comprises: a first compartment in fluid communication with the outlet opening of the exhaust housing; a second compartment spaced proximally from the first compartment of the expiration pathway and positioned in alignment with the first compartment of the expiration pathway; and a support frame positioned between the first and second compartments of the expiration pathway, wherein the support frame permits airflow between the second and first compartments of the expiration pathway and comprises a projection that extends distally within the first compartment of the expiration pathway, wherein the second blocking component is secured to the projection of the expiration pathway, wherein, in response to inspiration through the inlet opening and the inspiration pathway of the exhaust housing, the first blocking component is configured to deform proximally within the second compartment of the inspiration pathway to permit increased airflow through the support frame of the inspiration pathway, and wherein, in response to expiration through the port of the exhaust housing, the second blocking component is configured to deform distally within the first compartment of the expiration pathway to permit airflow through the support frame of the expiration pathway.

Aspect 29: The device of aspect 28, wherein the support frame of the inspiration pathway has opposing proximal and distal surfaces, wherein the projection of the support frame of the inspiration pathway extends proximally from the proximal surface, and wherein the first blocking component is biased toward a resting position in which the first blocking component abuts at least a portion of the proximal surface of the support frame of the inspiration pathway and sufficiently overlies the support frame to prevent airflow from the first compartment of the inspiration pathway to the second compartment of the inspiration pathway, and wherein the support frame of the expiration pathway has opposing proximal and distal surfaces, wherein the projection of the support frame of the expiration pathway extends distally from the distal surface, and wherein the second blocking component is biased toward a resting position in which the second blocking component abuts at least a portion of the distal surface of the support frame of the expiration pathway and sufficiently overlies the support frame to prevent airflow from the second compartment of the expiration pathway to the first compartment of the expiration pathway.

Aspect 30: The device of any one of aspecst 26-29, wherein the first and second blocking components comprise silicone discs.

Aspect 31: The device of any one of aspecst 26-30, wherein the first and second blocking components have respective thicknesses of less than 0.025 inches.

Aspect 32: The device of any one of aspecst 25-31, wherein the airflow adjustment plate is rotatably coupled to the exhaust housing using a fastener.

Aspect 33: The device of any one of aspecst 25-32, wherein the airflow adjustment plate comprises plastic.

Aspect 34: The device of any one of aspecst 25-33, wherein the airflow adjustment plate has a diameter that exceeds a maximum diameter of the exhaust housing.

Aspect 35: The device of any one of aspecst 25-34, wherein the airflow adjustment plate further defines a second set of openings, wherein the first and second sets of openings of the airflow adjustment plate are spaced along respective arcuate paths, and wherein at each rotational position, a respective opening of the second set of openings of the airflow adjustment plate is positioned in alignment with the outlet opening of the exhaust housing.

Aspect 36: The device of aspect 35, wherein the arcuate paths of the first and second sets of openings have differing radii of curvature.

Aspect 37: The device of any one of aspecst 35-36, wherein the first and second sets of openings of the airflow adjustment plate each comprise from 2 to 5 openings.

Aspect 38: The device of aspect 37, wherein the openings of the second set of openings of the airflow adjustment plate have equal diameters.

Aspect 39: The device of aspect 38, wherein at least one opening of the first set of openings of the airflow adjustment plate has a diameter that is less than the diameter of each opening of the second set of openings of the airflow adjustment plate.

Aspect 40: The device of any one of aspecst 25-39, wherein each opening of the first set of openings of the airflow adjustment plate has a different diameter than each other opening of the first set of openings.

Aspect 41: The device of any one of aspecst 25-40, wherein each opening of the first set of openings of the airflow adjustment plate has a different area than each other opening of the first set of openings.

Aspect 42: The device of aspect 41, wherein the first set of openings of the airflow adjustment plate comprises three openings, wherein a first opening of the first set of openings has an area corresponding to about 100% of an area of the inlet opening of the exhaust housing, wherein a second opening of the first set of openings has an area corresponding to about 50% of the area of the inlet opening of the exhaust housing, and wherein a third opening of the first set of openings has an area corresponding to about 25% of the area of the inlet opening of the exhaust housing.

Aspect 43: The device of any one of aspecst 25-42, wherein at least one of the airflow adjustment plate or the exhaust housing is configured to provide a tactile or audible indication in response to movement among the plurality of rotational positions of the airflow adjustment plate.

Aspect 44: The device of any one of aspecst 25-43, further comprising a patient interface component that is configured for engagement with the port of the exhaust housing.

Aspect 45: The device of aspect 44, wherein the patient interface component defines a port opening, wherein the port opening is configured for positioning in fluid communication with the port of the exhaust housing such that the exhaust housing frictionally engages the patient interface component.

Aspect 46: The device of aspect 44 or aspect 45, wherein the patient interface component comprises a strap.

Aspect 47: The device of aspect 44 or aspect 45, further comprising a strap that is configured to selectively secure the patient interface component to a subject.

Aspect 48: The device of any one of aspecst 25-47, wherein the device is disposable.

Aspect 49: The device of aspect 44, wherein the patient interface component is a mouthpiece that is configured for engagement with the port of the exhaust housing.

Aspect 50: The device of aspect 49, further comprising a nose clip.

Aspect 51: A kit comprising: an exhaust housing having a proximal portion that defines a port and an opposed distal portion that defines respective inspiration and expiration pathways, wherein the distal portion has a distal end surface, and wherein the inspiration and expiration pathways are in fluid communication with the port and extend, respectively, to inlet and outlet openings defined in the distal end surface; and a plurality of airflow adjustment plates configured to be rotatably coupled to the distal portion of the exhaust housing, wherein each airflow adjustment plate defines a first set of openings, wherein the first set of openings of each airflow adjustment plate comprises a plurality of openings having varying sizes, and wherein the first set of openings of each airflow adjustment plate differ from the first set of openings of each other airflow adjustment plate in diameter, number, or area, and wherein following rotatable coupling of a respective airflow adjustment plate to the distal portion of the exhaust housing, rotation of the airflow adjustment plate relative to the exhaust housing among a plurality of rotational positions increases or decreases resistance to air flow through the inlet opening of the exhaust housing, wherein at each rotational position, a respective opening of the first set of openings of the airflow adjustment plate is positioned in alignment with the inlet opening of the exhaust housing.

Aspect 52: A method of using the device of any one of aspecst 25-50 or 58-64.

Aspect 53: The method of claim 52, comprising: operatively positioning the port of the exhaust housing of the device relative to a subject; and using the device to deliver inspiratory air to the subject in response to inspiration of the subject that exceeds a cracking pressure threshold of a first blocking component positioned within the inspiration pathway of the exhaust assembly, wherein in response to exceeding the cracking pressure threshold, the first blocking component flexes from a resting position to an inspiration position that permits airflow from the inlet opening of the exhaust housing to the port of the exhaust housing.

Aspect 54: The method of aspect 53, further comprising: using the device to receive expired air from the subject, wherein in response to expiration by the subject, the first blocking component returns to the resting position and a second blocking component positioned within the expiration pathway of the exhaust assembly flexes from a resting position to an expiration position that permits airflow from the port of the exhaust housing to the outlet opening of the exhaust housing.

Aspect 55: The method of any one of aspecst 52-54, further comprising rotating the airflow adjustment plate to selectively adjust a threshold respiratory effort of the device.

Aspect 56: The method of any one of aspecst 52-55, further comprising capturing an echocardiogram of the subject during use of the device.

Aspect 57: The method of aspect 56, further comprising using the echocardiogram to determine an estimate of right atrium pressure (RAP) of the subject.

Aspect 58: The device of any one of aspecst 25-34, wherein the airflow adjustment plate further defines at least one second opening, wherein the first sets of openings and the at least one second opening of the airflow adjustment plate are spaced along respective arcuate paths, and wherein at each rotational position, the at least one second opening of the airflow adjustment plate is positioned in alignment with the outlet opening of the exhaust housing.

Aspect 59: The device of aspect 58, wherein the arcuate paths of the first set of openings and the at least one second opening have differing radii of curvature.

Aspect 60: The device of aspect 58 or aspect 59, wherein the first sets of openings of the airflow adjustment plate comprises from 2 to 5 openings.

Aspect 61: The device of aspect 60, wherein the at least one second opening exposes an equal area of the outlet opening of the exhaust housing in each of the plurality of plurality of rotational positions.

Aspect 62: The device of aspect 61, wherein at least one opening (optionally, each) of the first set of openings of the airflow adjustment plate exposes an area of the inlet opening of the exhaust housing that is less than the area of the outlet opening of the exhaust housing that the at least one second opening exposes.

Aspect 63: The device of any one of aspecst 25-50 or 58-62, wherein the first set of openings comprises a first opening, a second opening, and a third opening, wherein each of the first, second and third openings cooperates with the first blocking component to cause a corresponding threshold inspiratory pressure, wherein the threshold inspiratory pressures associated with the first and second openings are less than 5 mm Hg.

Aspect 64: The device of aspect 63, wherein the threshold inspiratory pressures associated with the first and second openings are less than 4 mm Hg.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be included within the scope of the appended claims. Moreover, although specific terms are employed herein, as well as in the claims which follow, they are used only in a generic and descriptive sense, and not for the purposes of limiting the described invention, nor the claims which follow.

What is claimed is:

1. A device having a longitudinal axis and comprising:
an exhaust assembly having:
an exhaust housing having a proximal portion that defines a port and an opposed distal portion that defines respective inspiration and expiration pathways, wherein the distal portion has a distal end surface, and wherein the inspiration and expiration pathways are in fluid communication with the port and extend, respectively, to inlet and outlet openings defined in the distal end surface; and
an airflow adjustment plate rotatably coupled to the distal portion of the exhaust housing, wherein the airflow adjustment plate defines a first set of openings, and wherein the first set of openings of the airflow adjustment plate comprises a plurality of openings having varying sizes, and
wherein rotation of the airflow adjustment plate relative to the exhaust housing among a plurality of rotational positions increases or decreases resistance to air flow through the inlet opening of the exhaust housing, wherein at each rotational position, a respective opening of the first set of openings of the airflow adjustment plate is positioned in alignment with the inlet opening of the exhaust housing.

2. The device of claim 1, further comprising:
a first blocking component positioned within the inspiration pathway of the exhaust housing; and
a second blocking component positioned within the expiration pathway of the exhaust housing,
wherein, in response to inspiration through the inlet opening and the inspiration pathway of the exhaust housing, the first blocking component is configured to deform to permit increased airflow to the port of the exhaust housing, and the second blocking component is configured to prevent airflow through the expiration pathway, and
wherein, in response to expiration through the port of the exhaust housing, the second blocking component is configured to deform to permit airflow to the outlet opening of the exhaust housing, and the first blocking component is configured to restrict airflow through the inspiration pathway.

3. The device of claim 2, wherein the inspiration pathway comprises:
a first compartment in fluid communication with the inlet opening of the exhaust housing;
a second compartment spaced proximally from the first compartment and positioned in alignment with the first compartment; and
a support frame positioned between the first and second compartments, wherein the support frame permits airflow through the inspiration pathway and comprises a projection that extends proximally within the second compartment,
wherein the first blocking component is secured to the projection.

4. The device of claim 3, wherein the expiration pathway comprises:
a first compartment in fluid communication with the outlet opening of the exhaust housing;
a second compartment spaced proximally from the first compartment of the expiration pathway and positioned in alignment with the first compartment of the expiration pathway; and
a support frame positioned between the first and second compartments of the expiration pathway, wherein the support frame permits airflow between the second and first compartments of the expiration pathway and comprises a projection that extends distally within the first compartment of the expiration pathway,
wherein the second blocking component is secured to the projection of the expiration pathway,
wherein, in response to inspiration through the inlet opening and the inspiration pathway of the exhaust housing, the first blocking component is configured to deform proximally within the second compartment of the inspiration pathway to permit increased airflow through the support frame of the inspiration pathway, and
wherein, in response to expiration through the port of the exhaust housing, the second blocking component is configured to deform distally within the first compartment of the expiration pathway to permit airflow through the support frame of the expiration pathway.

5. (canceled)

6. (canceled)

7. (canceled)

8. The device of claim 1, wherein the airflow adjustment plate is rotatably coupled to the exhaust housing using a fastener.

9. (canceled)

10. The device of claim 1, wherein the airflow adjustment plate has a diameter that exceeds a maximum diameter of the exhaust housing.

11. The device of claim 1, wherein the airflow adjustment plate further defines at least one second opening, wherein the first sets of openings and the at least one second opening of the airflow adjustment plate are spaced along respective arcuate paths, and wherein at each rotational position, the at least one second opening of the airflow adjustment plate is positioned in alignment with the outlet opening of the exhaust housing.

12. The device of claim 11, wherein the arcuate paths of the first set of openings and the at least one second opening have differing radii of curvature.

13. The device of claim 11, wherein the first sets of openings of the airflow adjustment plate comprises from 2 to 5 openings.

14. The device of claim 13, wherein the at least one second opening exposes an equal area of the outlet opening of the exhaust housing in each of the plurality of plurality of rotational positions.

15. The device of claim 14, wherein at least one opening of the first set of openings of the airflow adjustment plate exposes an area of the inlet opening of the exhaust housing that is less than the area of the outlet opening of the exhaust housing that the at least one second opening exposes.

16. The device of claim 1, wherein each opening of the first set of openings of the airflow adjustment plate has a different diameter than each other opening of the first set of openings.

17. The device of claim 1, wherein each opening of the first set of openings of the airflow adjustment plate has a different area than each other opening of the first set of openings.

18. The device of claim 17, wherein the first set of openings of the airflow adjustment plate comprises three openings, wherein a first opening of the first set of openings has an area corresponding to about 100% of an area of the inlet opening of the exhaust housing, wherein a second opening of the first set of openings has an area corresponding to about 50% of the area of the inlet opening of the exhaust housing, and wherein a third opening of the first set of openings has an area corresponding to about 25% of the area of the inlet opening of the exhaust housing.

19. The device of claim 1, wherein at least one of the airflow adjustment plate or the exhaust housing is configured to provide a tactile or audible indication in response to movement among the plurality of rotational positions of the airflow adjustment plate.

20. The device of claim 1, further comprising a patient interface component that is configured for engagement with the port of the exhaust housing.

21. The device of claim 20, wherein the patient interface component defines a port opening, wherein the port opening is configured to be positioned in fluid communication with the port of the exhaust housing such that the exhaust housing frictionally engages the patient interface component.

22. (canceled)

23. (canceled)

24. (canceled)

25. The device of claim 20, wherein the patient interface component is a mouthpiece that is configured for engagement with the port of the exhaust housing.

26. (canceled)

27. The device of claim 1, wherein the first set of openings comprises a first opening, a second opening, and a third opening, wherein each of the first, second and third openings cooperates with the first blocking component to cause a corresponding threshold inspiratory pressure, wherein the threshold inspiratory pressures associated with the first and second openings are less than 5 mm Hg.

28. (canceled)

29. A kit comprising:
an exhaust housing having a proximal portion that defines a port and an opposed distal portion that defines respective inspiration and expiration pathways, wherein the distal portion has a distal end surface, and wherein the inspiration and expiration pathways are in fluid communication with the port and extend, respectively, to inlet and outlet openings defined in the distal end surface; and
a plurality of airflow adjustment plates configured to be rotatably coupled to the distal portion of the exhaust housing, wherein each airflow adjustment plate defines a first set of openings, wherein the first set of openings of each airflow adjustment plate comprises a plurality of openings having varying sizes, and wherein the first set of openings of each airflow adjustment plate differ from the first set of openings of each other airflow adjustment plate in diameter, number, or area, and
wherein following rotatable coupling of a respective airflow adjustment plate to the distal portion of the exhaust housing, rotation of the airflow adjustment plate relative to the exhaust housing among a plurality of rotational positions increases or decreases resistance to air flow through the inlet opening of the exhaust housing, wherein at each rotational position, a respective opening of the first set of openings of the airflow adjustment plate is positioned in alignment with the inlet opening of the exhaust housing.

30. A method comprising:
operatively positioning a port of an exhaust housing of a device relative to a subject, wherein the device comprises:
an exhaust assembly having:
the exhaust housing, wherein the exhaust housing has a proximal portion that defines the port and an opposed distal portion that defines respective inspiration and expiration pathways, wherein the distal portion has a distal end surface, and wherein the inspiration and expiration pathways are in fluid communication with the port and extend, respectively, to inlet and outlet openings defined in the distal end surface; and
an airflow adjustment plate rotatably coupled to the distal portion of the exhaust housing, wherein the airflow adjustment plate defines a first set of openings, and wherein the first set of openings of the airflow adjustment plate comprises a plurality of openings having varying sizes,
wherein rotation of the airflow adjustment plate relative to the exhaust housing among a plurality of rotational positions increases or decreases resistance to air flow through the inlet opening of the exhaust housing, wherein at each rotational position, a respective opening of the first set of openings of the airflow adjustment plate is positioned in alignment with the inlet opening of the exhaust housing; and
using the device to deliver inspiratory air to the subject in response to inspiration of the subject that exceeds a cracking pressure threshold of a first blocking component positioned within the inspiration pathway of the exhaust assembly, wherein in response to exceeding the cracking pressure threshold, the first blocking component flexes from a resting position to an inspiration position that permits airflow from the inlet opening of the exhaust housing to the port of the exhaust housing.

31. (canceled)

32. (canceled)

33. (canceled)

34. (canceled)
